(19) United States
(12) Patent Application Publication
(10) Pub. No.: US 2013/0242052 A1
(43) Pub. Date: Sep. 19, 2013

(54) IMAGE PROCESSING DEVICE AND IMAGE PROCESSING METHOD

(71) Applicant: SONY CORPORATION, Tokyo (JP)

(72) Inventor: Tsuneo Hayashi, Chiba (JP)

(73) Assignee: Sony Corporation, Tokyo (JP)

(21) Appl. No.: 13/777,165

(22) Filed: Feb. 26, 2013

(30) Foreign Application Priority Data
Mar. 16, 2012 (JP) 2012-059736

(51) Int. Cl. H04N 13/02

(52) U.S. Cl. CPC H04N 13/02
USPC 348/45

(57) ABSTRACT

An image processing device which includes an image selection unit which selects a viewpoint image according to a viewpoint rotation angle from a plurality of viewpoint images having different viewpoints, and an addition processing unit which generates a viewpoint image with a new viewpoint by adding a viewpoint image which is selected in the image selection unit.

### BACKGROUND

The present technology relates to an image processing device, and an image processing method, and is a technology in which a direction of a stereoscopic vision can be freely, and easily changed.

In the related art, an endoscope has been widely used in order to observe the inside of a pipe, or a body cavity. As the endoscope, there are a flexible endoscope which can observe the inside by inserting a flexible insertion unit into a bent pipe, a body cavity, or the like, and a rigid endoscope which can observe the inside by linearly inserting a rigid insertion unit into a target portion.

As the flexible endoscope, for example, there is an optical endoscope in which an optical image which is imaged using an imaging optical system on the tip end is transmitted to an eyepiece unit using an optical fiber, or an electronic endoscope in which an optical image of a subject which is imaged using an imaging optical system by providing the imaging optical system and an imaging element on the tip end is transmitted to an external monitor by being converted into an electric signal using the imaging element. In the rigid endoscope, an optical image of a subject is transmitted to an eyepiece unit using a relay optical system which is configured by linking a lens system from the tip end.

Further, as the endoscope, a stereoscopic vision endoscope has been commercialized in order to easily observe a minute irregularity on the inner wall surface in a pipe, a body cavity, or the like. For example, in Japanese Unexamined Patent Application Publication No. 06-059199, an optical image of a subject which is transmitted using a relay optical system is divided into a left subject optical image and a right subject optical image around the optical axis of the relay optical system using a pupil division prism. Further, the left subject optical image and right subject optical image which are divided using the pupil division prism are converted to an image signal using an imaging element, respectively. In addition, the pupil division prism and the two imaging elements are rotated around the optical axis of the relay optical system using a rotation mechanism. It is possible to freely change the direction in the stereoscopic vision without moving an endoscope by configuring the endoscope in this manner.

### SUMMARY

Meanwhile, when adopting a configuration in which an optical image of a subject is divided into a left subject optical image and a right subject optical image around an optical axis of a relay optical system using a pupil division prism, or a configuration in which the pupil division prism and two imaging elements are rotated around the optical axis of the relay optical system, an optical system of an endoscope or the like becomes large, and it is difficult to perform miniaturization. In addition, there is a concern that a malfunction or the like may easily occur due to the rotation of image, since the pupil division prism and two imaging elements are mechanically rotated. In addition, it is difficult to perform an adjustment easily, and with high precision, since a mechanical rotation mechanism is used. In addition, in order to compensate an assembling error, a deterioration due to time, a change in temperature, or the like, calibration is used.

It is desirable to provide an image processing device, an image processing method, and a program thereof which can freely and easily change a direction of a stereoscopic vision.

According to a first embodiment of the present technology, there is provided an image processing device which includes an image selection unit which selects a viewpoint image according to a viewpoint rotation angle from a plurality of viewpoint images having different viewpoints, and an addition processing unit which generates a viewpoint image with a new viewpoint by adding a viewpoint image which is selected in the image selection unit.

In the technology, a plurality of viewpoint images having different viewpoints, for example, a plurality of viewpoint images having different viewpoints are generated from light beam information including channel information of a light beam which is input through an imaging optical system of an imaging unit, and light quantity information of a light beam. In addition, a viewpoint image of a viewpoint which is included in a plurality of viewpoint regions which are set according to a viewpoint rotation angle, for example, a viewpoint region of a left eye image, and a viewpoint region of a right eye image is selected from the plurality of viewpoint images having different viewpoints in each region in the image selection unit. The viewpoint image which is selected in each region is added in each region in the addition processing unit, and a viewpoint image having a new viewpoint, for example, a left eye image, and a right eye image are generated. In addition, all of the viewpoint images are selected, or viewpoint images with viewpoints included in viewpoint regions of the left eye image and right eye image are selected, thereby generating a plan image by adding the selected viewpoint images. Further, by controlling a gap between viewpoint region of a left eye image and a viewpoint region of a right eye image, a parallax amount of the left eye image and right eye image is adjusted.

A gain adjustment corresponding to the number of viewpoint images which is added with respect to a viewpoint image with a new viewpoint image which is generated by adding a viewpoint image, that is, a gain adjustment in which gain is set to be high when the number of added viewpoint images is small, and an influence due to a difference in the number of added viewpoint images is excluded. In addition, a direction of a viewpoint image with a new viewpoint is determined according to a viewpoint rotation angle by performing image rotation processing according to the viewpoint rotation angle.

When setting a viewpoint rotation angle, for example, an angle of an imaging unit with respect to any one of the gravity direction and the initial direction, an angle in which an image which is imaged in the imaging unit becomes an image which is the most similar to a reference image when being rotated, or an angle which is designated by a user is set to the viewpoint rotation angle. In addition, a viewpoint image with a new viewpoint is generated by providing an image decoding unit which performs the decoding processing of an encoding signal which is generated by performing encoding processing of a plurality of viewpoint images having different viewpoints, and using image signals of the plurality of viewpoint images having different viewpoints which are obtained by performing decoding processing of the encoding signal.

According to a second embodiment of the present technology, there is provided an image processing method which includes selecting a viewpoint image according to a viewpoint rotation angle from a plurality of viewpoint images having different viewpoints, and generating a viewpoint image with a new viewpoint by adding the selected viewpoint image.

According to the present technology, a viewpoint image with a new viewpoint is generated by selecting a viewpoint image according to a viewpoint rotation angle from a plurality of viewpoint images having different viewpoints, and adding the selected viewpoint image. Accordingly, when the viewpoint rotation angle is changed, it is possible to easily and freely change the direction of a stereoscopic vision by generating a left eye image and right eye image by adding the selected viewpoint image which is selected according to the viewpoint rotation angle.

### BRIEF DESCRIPTION OF THE DRAWINGS

FIGS. 1A to 1C are diagrams which illustrate endoscopes;

FIG. 2 is a diagram which illustrates a configuration example of an endoscope device to which an image processing device is applied;

FIG. 3 is a diagram which illustrates a configuration example of a light field camera;

FIG. 4 is an explanatory diagram of a plurality of viewpoint images;

FIGS. 5A and 5B are diagrams which exemplify an arrangement of a viewpoint;

FIG. 6 is a diagram which illustrates a configuration example of an image processing unit of viewpoint 1;

FIG. 7 is a diagram which illustrates a configuration example of an image selection unit;

FIG. 8 is a diagram which illustrates a configuration example of a viewpoint rotation angle setting unit;

FIG. 9 is a flowchart which illustrates a part of image processing operation in an endoscope;

FIGS. 10A to 10D are diagrams which exemplify a relationship between a rotation angle and a viewpoint image which is selected in the image selection unit (when number of viewpoints is “256”);

FIGS. 11A to 11D are diagrams which exemplify a relationship between a rotation angle and a viewpoint image which is selected in the image selection unit (when number of viewpoints is “16”);

FIG. 12 is a diagram which illustrates a configuration example of an endoscope;

FIG. 13 is a flowchart which illustrates a part of an operation of an endoscope;

FIG. 14 is a diagram which illustrates a configuration example of an image processing device;

FIG. 15 is a flowchart which exemplifies an operation of the image processing device;

FIGS. 16A to 16C are diagrams which exemplify operations when a viewpoint is rotated in the horizontal direction;

FIGS. 17A to 17C are diagrams which exemplify operations when a parallax adjustment is performed;

FIG. 18 is a diagram when a viewpoint is set to four groups;

FIG. 19 is a diagram when a viewpoint is set to eight groups; and

FIG. 20 is a diagram which illustrates a 2D addition processing unit.

### DETAILED DESCRIPTION OF EMBODIMENTS

Hereinafter, embodiments of the present technology will be described. In addition, descriptions will be made in the following order.

1. First Embodiment

2. Second Embodiment

3. Other Embodiments

### 1. First Embodiment

### 1-1. Appearance of Endoscope

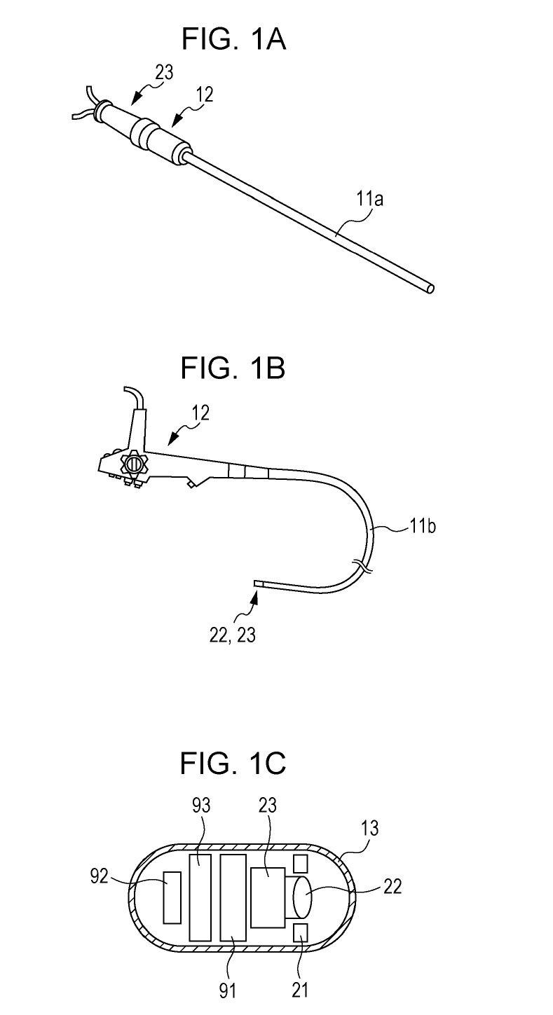

FIGS. 1A to 1C illustrate an endoscope. FIG. 1A is an appearance of a rigid endoscope, FIG. 1B illustrates an appearance of a flexible endoscope, and FIG. 1C illustrates an internal configuration of a capsule endoscope.

The rigid endoscope includes an insertion unit 11a which is inserted into an observation target, a grip portion 12 which is gripped by a user, and an imaging unit 23. The insertion unit 11a includes an image guide shaft, and light guiding fiber. Light which is emitted from a light source unit to be described later is radiated to an observation target through an imaging lens which is provided at the tip end of the light guiding fiber and the insertion unit 11a. In addition, subject light from the observation target is input to the imaging unit 23 through the imaging lens, and a relay optical system in the image guide shaft.

Similarly to the rigid endoscope, the flexible endoscope also includes the insertion unit 11b which is inserted into an observation target, a grip portion 12 which is gripped by a user, and an imaging unit 23. The insertion unit 11b of the flexible endoscope is flexible, and is provided with an imaging optical system 22, or the imaging unit 23 on the tip end.

The capsule endoscope is provided with, for example, a light source unit 21, an imaging optical system 22, an imaging unit 23, a processing unit 91 which performs various signal processes to be described later, a wireless communication unit 92 for performing transmitting of an image signal or the like after processing, a power source unit 93, or the like, in a housing 13.

### 1-1. Configuration of Endoscope Device

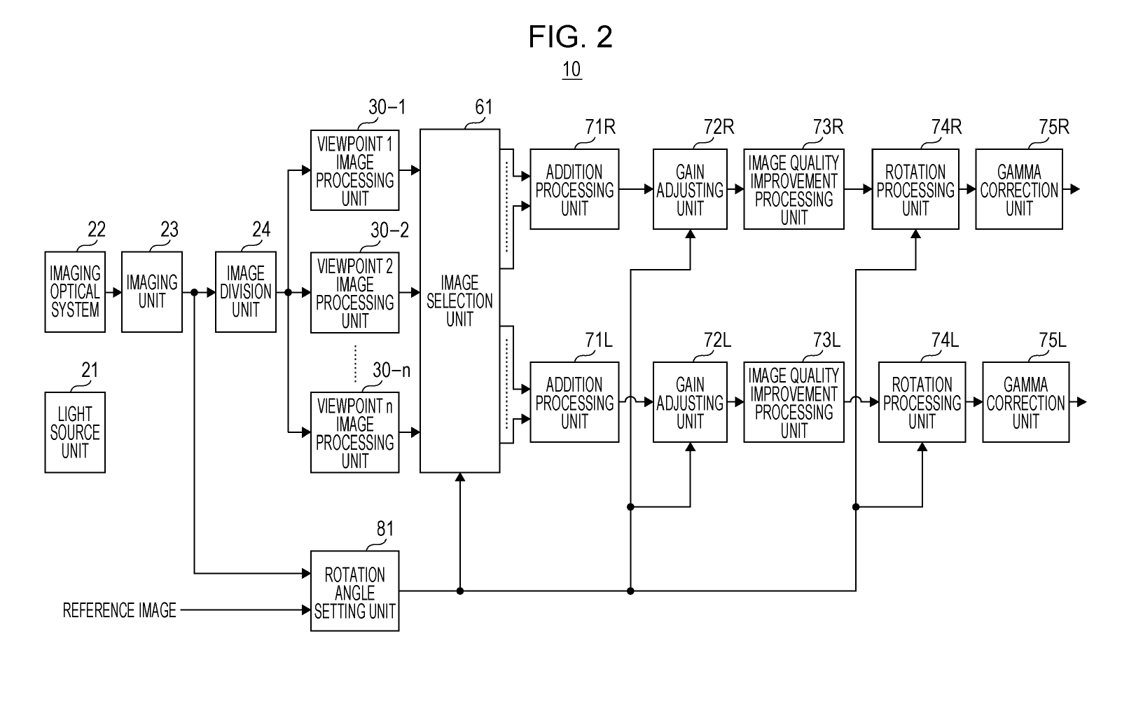

FIG. 2 illustrates a configuration example of an endoscope device to which an image processing device according to the embodiment of the present technology is applied. An endoscope device 10 includes a light source unit 21, an imaging optical system 22, an imaging unit 23, an image division unit 24, image processing units 30-1 to 30-n of viewpoints 1 to n, and an image selection unit 61. The endoscope device 10 further includes addition processing units 71L and 71R, gain adjustment units 72L and 72R, image quality improving processing units 73L and 73R, rotation processing units 74L and 74R, gamma correction units 75L and 75R, and a viewpoint rotation angle setting unit 81.

The light source unit 21 emits illumination light to an observation target. The imaging optical system 22 is configured by a focus lens, a zoom lens or the like, and causes an optical image of the observation target to which the illumination light is radiated (subject optical image) to be formed as an image in the imaging unit 23.

In the imaging unit 23, a light field camera which is able to record light beam information (light field data) which also includes channel information (direction of input light) of input light, not only light quantity information of the input light is used.

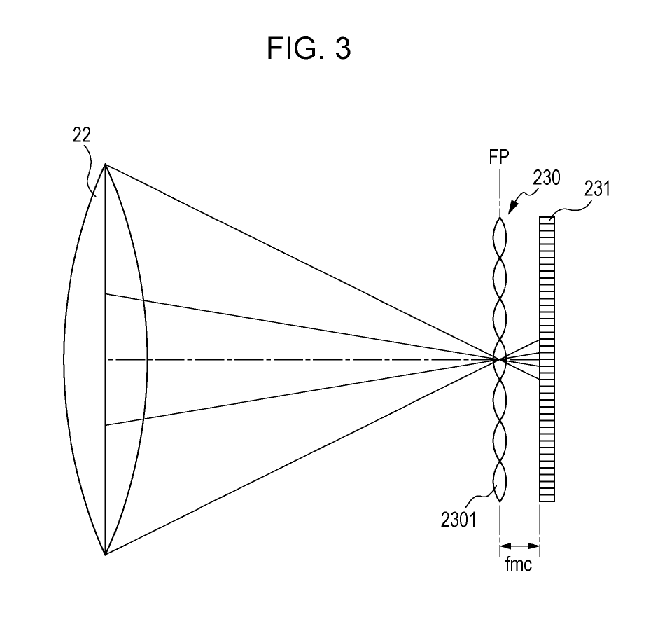

FIG. 3 illustrates a configuration example of a light field camera. The light field camera is provided with a microlens array 230 in the immediate front of an image sensor 231 such as a CCD (Charge Coupled Device), a CMOS (Complementary Metal Oxide Semiconductor), or the like.

The microlens array 230 is installed at a position of a focal plane FP of an imaging optical system 22. In addition, the position of the imaging optical system 22 is set to a distance which is considered to be in infinity with respect to the microlens of the microlens array 230. The image sensor 231 is installed so that a sensor plane thereof is located at the rear side (opposite side to imaging optical system 22) from the microlens array 230 by a focal length fmc of the microlens. Each microlens 2301 of the image sensor 231 and the microlens array 230 is configured so that a plurality of pixels of the image sensor 231 are included with respect to each microlens 2301.

In the light field camera having such a configuration, a pixel position of input light which is input to pixels through the microlens 2301 is changed according to the input direction. Accordingly, by using the light field camera, it is possible to generate light beam information including the light quantity information and the channel information of input light.

In addition, since the light field camera is configured so that the plurality of pixels of the image sensor 231 are included with respect to each microlens 2301, it is possible to obtain a plurality of viewpoint images having different viewpoint positions.

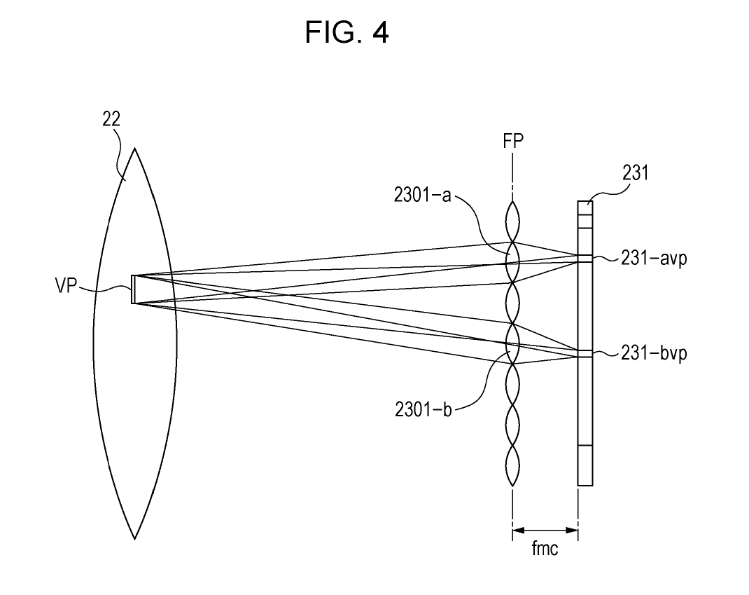

FIG. 4 is an explanatory diagram regarding a plurality of viewpoint images. When a viewpoint image is generated using light beam information, a relationship between a viewpoint and a pixel is calculated in advance in each microlens. For example, to which pixel input light which is input to the micro lens 2301-a through a viewpoint VP in the imaging optical system 22 is input, is calculated (in FIG. 4, a case of inputting to pixel 231-avp is illustrated). Similarly, to which pixel input light is input, is also calculated for input light which is input to the microlens 2301-b through the viewpoint VP (in FIG. 4, a case of inputting to pixel 231-bvp is illustrated). In addition, for other microlenses 2301, a pixel position to which input light which passes through a viewpoints VP is input, is additionally calculated in advance. In this manner, it is possible to generate the viewpoint image of the viewpoint VP by reading out a pixel signal of a pixel corresponding to the viewpoint VP in each microlens 2301, when calculating to which pixel position the input light which is input to the microlens through the viewpoint VP is input.

Here, when 16×16 pixels are included per microlens, it is possible to obtain a pixel signal of “256” viewpoint images having different viewpoint positions with respect to one microlens. In addition, the number of microlenses is the same as the number of pixels in each viewpoint image, and for example, in a case of a microlens array of 1024×1024, each viewpoint image has pixels of 1024×1024 pixels, and the number of whole pixels of an imaging element becomes 16k×16k=256M.

Similarly, when 8×8 pixels are included per microlens, it is possible to obtain a pixel signal of “64” viewpoint images having different viewpoint positions with respect to one microlens. In addition, the number of microlenses is the same as the number of pixels in each viewpoint image, and for example, in a case of a microlens array of 1024×1024, each viewpoint image has pixels of 1024×1024 pixels, and the number of whole pixels of an imaging element becomes 8k×8k=64M.

When 4×4 pixels are included per microlens, viewpoint images of “16” having different viewpoint positions are obtained with respect to one microlens. In addition, the number of microlenses is the same as the number of pixels in each viewpoint image, and for example, in a case of a microlens array of 1024×1024, each viewpoint image has pixels of 1024×1024 pixels, and the number of whole pixels of an imaging element becomes 4k×4k=16M.

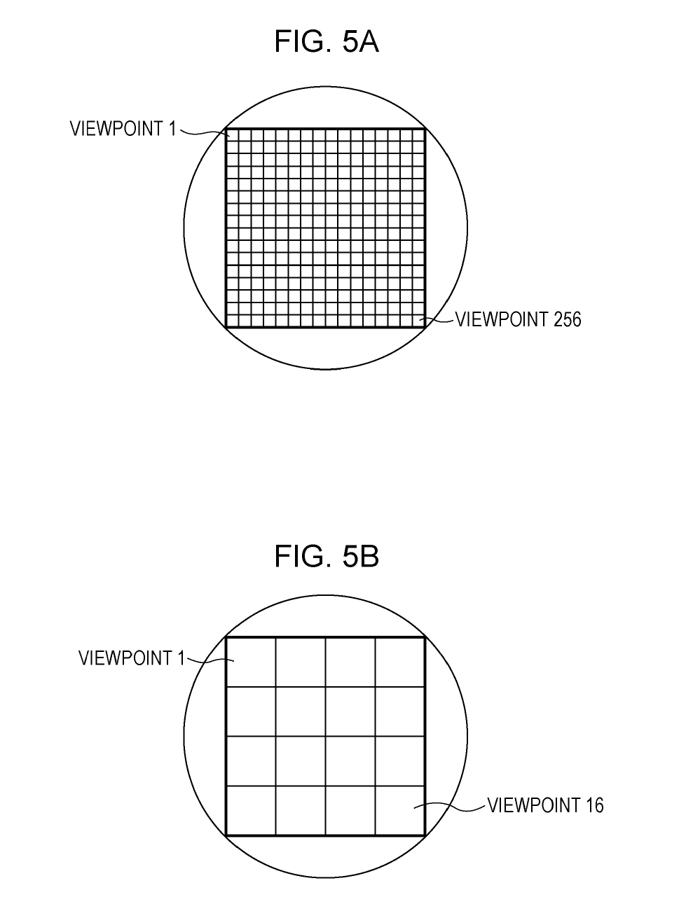

In addition, in an image processing operation in an endoscope which is described later, a case of 16×16 viewpoints (viewpoint 1 to viewpoint 256) as illustrated in FIG. 5A, and a case of 4×4 viewpoints (viewpoint 1 to viewpoint 4) as illustrated in FIG. 5B will be described.

The image division unit 24 divides light beam information which is generated in the imaging unit 23 in every viewpoint, and generates image signals of a plurality of viewpoint images. The image division unit 24 generates image signals of, for example, viewpoint 1 image to viewpoint n image. The image division unit 24 outputs an image signal of the viewpoint 1 image to a viewpoint 1 image processing unit 30-1. Similarly, the image division unit 24 outputs an image signal of the viewpoint 2 (to n) image to a viewpoint 2 (to n) image processing unit 30-2 (to n).

The viewpoint 1 image processing unit 30-1 to the viewpoint n image processing unit 30-n performs image processing with respect to image signals of viewpoint images which are supplied from the image division unit 24.

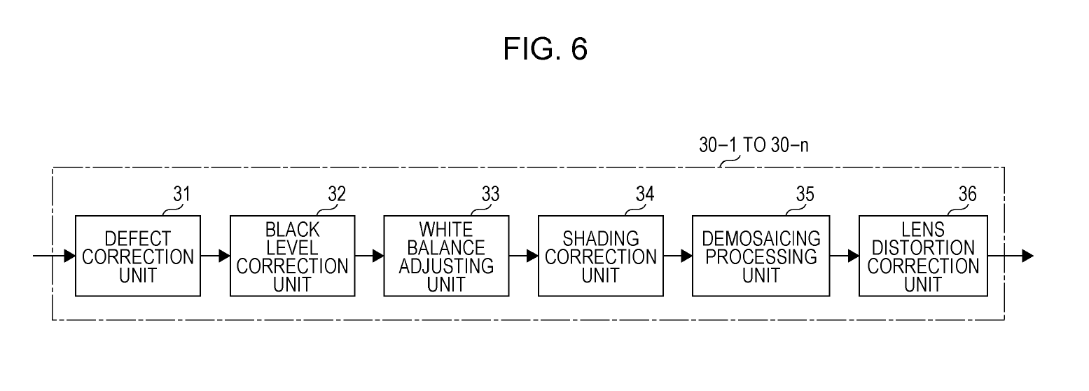

FIG. 6 illustrates a configuration example of the viewpoint 1 image processing unit. In addition, the viewpoint 2 image processing unit 30-2 to the viewpoint n image processing unit 30-n also have the same configuration as that of the viewpoint 1 image processing unit.

The viewpoint 1 image processing unit 30-1 includes a defect correction unit 31, a black level correction unit 32, a white balance adjusting unit 33, a shading correction unit 34, a demosaicing processing unit 35, and a lens distortion correction unit 36.

The defect correction unit 31 performs signal correction processing with respect to defective pixels of an imaging element, and outputs a corrected image signal to the black level correction unit 32. The black level correction unit 32 performs clamp processing in which a black level of an image signal is adjusted, and the image signal after the clamp processing is output to a white balance adjusting unit 33. The white balance adjusting unit 33 performs a gain adjustment of an image signal so that each color component of red, green, and blue of a white subject on an input image becomes the same as that in a white color. The white balance adjusting unit 33 outputs the image signal after the white balance adjustment to the shading correction unit 34.

The shading correction unit 34 corrects peripheral light quantity drop of a lens, and outputs an image signal after correcting to the demosaicing processing unit 35. The demosaicing processing unit 35 generates a signal with a color component of a pixel which is omitted in an intermittent arrangement by an interpolation using a pixel in the periphery thereof, that is, a signal of a pixel having a different space phase according to a color arrangement of a color filter which is used in the imaging unit 23. The demosaicing processing unit 35 outputs the image signal after the demosaicing processing to a lens distortion correction unit 36. The lens distortion correction unit 36 performs a correction of distortion or the like which occurs in the imaging optical system 22.

In this manner, the viewpoint 1 image processing unit 30-1 performs various correction processing, adjustment processing or the like, with respect to the image signal of the viewpoint 1 image, and outputs the image signal after processing to the image selection unit 61. In addition, the viewpoint 1 image processing unit 30-1 to the viewpoint n image processing unit 30-n may be configured using a different order, by adding another processing, or by eliminating a part of processes without being limited to the case of processing which is performed in the configuration order in FIG. 6.

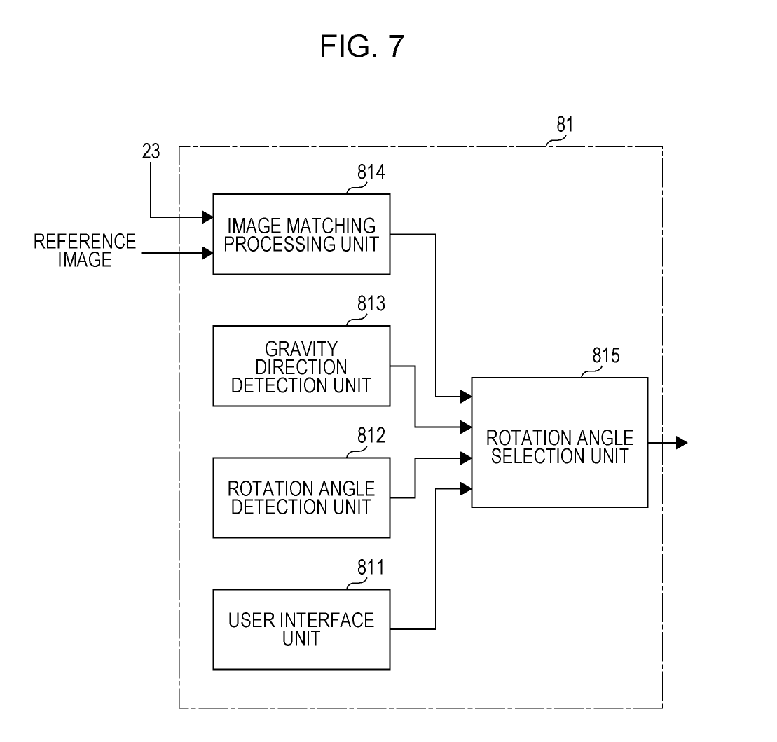

The image selection unit 61 selects a viewpoint image according to a viewpoint rotation angle from a plurality of viewpoint images having different viewpoints. The image selection unit 61 sets a plurality of viewpoint regions, for example, a viewpoint region of a left eye image and a viewpoint region of a right eye image based on the rotation angle which is set in the viewpoint rotation angle setting unit 81, and selects a viewpoint image of a viewpoint which is included in the set viewpoint region in each region. The image selection unit 61 outputs a viewpoint image with a viewpoint which is included in the viewpoint region of the left eye image to the addition processing unit 71L, and a viewpoint image with a viewpoint which is included in the viewpoint region of the right eye image to the addition processing unit 71R. As illustrated in FIG. 7, the image selection unit 61 includes an image selection table 611 and a matrix switching unit 612. The image selection table 611 stores image selection information corresponding to the rotation angle by making the information as a table. The image selection information is information for selecting the image signals of the viewpoint images which are added in the addition processing units 71L and 71R in the matrix switching unit 612 in order to generate the image signals of the left eye image and right eye image corresponding to the rotation angle in the addition processing units 71L and 71R. The image selection table 611 outputs image selection information corresponding to the rotation angle which is set in the viewpoint rotation angle setting unit 81 to the matrix switching unit 612. In addition, the image selection unit 61 may output the image selection information to the matrix switching unit 612 by calculating the image selection information in each setting of the rotation angle, without using the image selection table 611.

The matrix switching unit 612 performs switching based on the image selection information, selects an image signal of a viewpoint image for generating a left eye image corresponding to a rotation angle from image signals of the viewpoint 1 image to the viewpoint n image, and outputs the image signal to the addition processing unit 71L. In addition, the matrix switching unit 612 performs switching based on the image selection information, selects an image signal of a viewpoint image for generating a right eye image corresponding to a rotation angle from image signals of the viewpoint 1 image to the viewpoint n image, and outputs the image signal to the addition processing unit 71R.

Returning to FIG. 2, the addition processing unit 71L generates an image signal of a left eye image by adding the viewpoint image which is supplied from the image selection unit 61. The addition processing unit 71L outputs the generated image signal of the left eye image to a gain adjusting unit 72L. The addition processing unit 71R generates an image signal of the right eye image by adding the viewpoint image which is supplied from the image selection unit 61. The addition processing unit 71R outputs the generated image signal of the right eye image to the gain adjusting unit 72R.

The gain adjusting unit 72L performs gain adjusting corresponding to a rotation angle with respect to the image signal of the left eye image. The image signal of the left eye image is generated by adding the image signal which is selected in the image selection unit 61 in the addition processing unit 71L. Accordingly, when the number of viewpoint images which are selected in the image selection unit 61 is small, a signal level of the image signal becomes small. Accordingly, the gain adjusting unit 72L performs gain adjusting according to the number of viewpoint images which are added when generating image signal of the left eye image, and removes an influence due to the difference in the number of added viewpoint images. The gain adjusting unit 72L outputs the image signal after the gain adjusting to an image quality improvement processing unit 73L.

The gain adjusting unit 72R performs the same gain adjusting corresponding to a rotation angle with respect to an image signal of a right eye image as that in the gain adjusting unit 72L, performs gain adjusting according to the number of added viewpoint images, and removes an influence due to the difference in the number of added viewpoint images. A gain adjusting unit 72R outputs the image signal after the gain adjusting to an image quality improvement processing unit 73R.

The image quality improvement processing unit 73L performs high resolution of an image using classification adaptation processing or the like. For example, the image quality improvement processing unit 73L generates an image signal with high resolution by improving sharpness, contrast, color, or the like. The image quality improvement processing unit 73L outputs the image signal after the image quality improvement processing to a rotation processing unit 74L.

The image quality improvement processing unit 73R performs high resolution of an image using classification adaptation processing or the like, similarly to the image quality improvement processing unit 73L, and outputs the image signal after the image quality improvement processing to a rotation processing unit 74R.

The rotation processing unit 74L performs a rotation of the left eye image. The rotation processing unit 74L performs rotation processing based on a rotation angle, and rotates the direction of the generated left eye image. The rotation processing unit 74L outputs the image signal of the left eye image after rotating to a gamma correction unit 75L. The rotation processing unit 74R performs a rotation of the right eye image. The rotation processing unit 74R performs rotation processing based on a rotation angle, and rotates the direction of the generated right eye image. The rotation processing unit 74R outputs the image signal of the right eye image after rotating to a gamma correction unit 75R.

The gamma correction unit 75L performs correction processing based on gamma characteristics of the display device performing an image display of an imaged image with respect to the left eye image, and outputs an image signal of the left eye image which is subjected to the gamma correction to the display device or the like. The gamma correction unit 75R performs correction processing based on gamma characteristics of the display device performing an image display of an imaged image with respect to the right eye image, and outputs an image signal of the right eye image which is subjected to the gamma correction to the display device or the like.

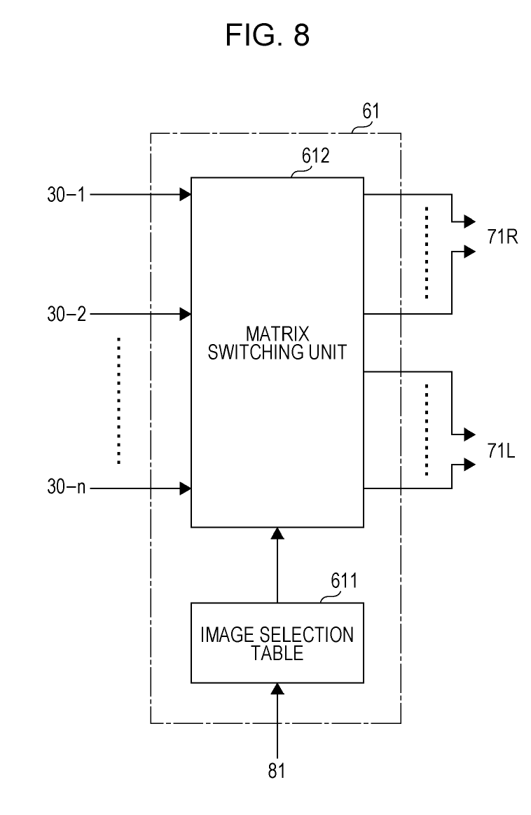

The viewpoint rotation angle setting unit 81 sets a viewpoint rotation angle at the time of generating a left eye image and a right eye image. FIG. 8 illustrates a configuration example of the viewpoint rotation angle setting unit. The viewpoint rotation angle setting unit 81 includes a user interface unit 811, a rotation angle detection unit 812, a gravity direction detection unit 813, an image matching processing unit 814, and a rotation angle selection unit 815.

The user interface (I/F) unit 811 is configured using an operation switch or the like, and outputs a rotation angle which is set by a user operation to the rotation angle selection unit 815.

The rotation angle detection unit 812 detects a rotation angle with respect to an initial position. The rotation angle detection unit 812 includes, for example, an angle sensor such as a gyro sensor, detects a rotation angle of the imaging unit 23 from the initial position using the angle sensor, and outputs the detected rotation angle to the rotation angle selection unit 815.

The gravity direction detection unit 813 detects the gravity direction. The gravity direction detection unit 813 is configured using, for example, a clinometer, an accelerometer, or the like, and detects the gravity direction. In addition, the gravity direction detection unit 813 outputs an angle of the imaging unit 23 with respect to the gravity direction to the rotation angle selection unit 815 as a rotation angle.

The image matching processing unit 814 generates a 2D imaged image using light beam information which is generated in the imaging unit 23. In addition, the image matching processing unit 814 performs subject detection with respect to the generated imaged image, and a reference image which is supplied from an external device, or the like. Further, the image matching processing unit 814 outputs a rotation angle in which a desired subject which is detected from the imaged image becomes closest to the position of the desired subject which is detected from the reference image by rotating the imaged image to the rotation angle selection unit 815.

The rotation angle selection unit 815 sets a rotation angle by selecting a rotation angle according to, for example, a user operation, or an operation setting of an endoscope from a supplied rotation angle. The viewpoint rotation angle setting unit 81 informs the image selection unit 61, and the rotation processing units 74L and 74R of the set rotation angle.

In addition, the configuration of the endoscope device is not limited to the configuration which is illustrated in FIG. 2, and, for example, may be a configuration in which the image quality processing unit is not provided. In addition, also the processing order is not limited to the configuration illustrated in FIG. 2, and it is also possible to perform the rotation processing before the gain adjusting, for example. In addition, the same is applied to an image processing unit 50 which will be described later.

### 1-2. Image Processing Operation in Endoscope Device

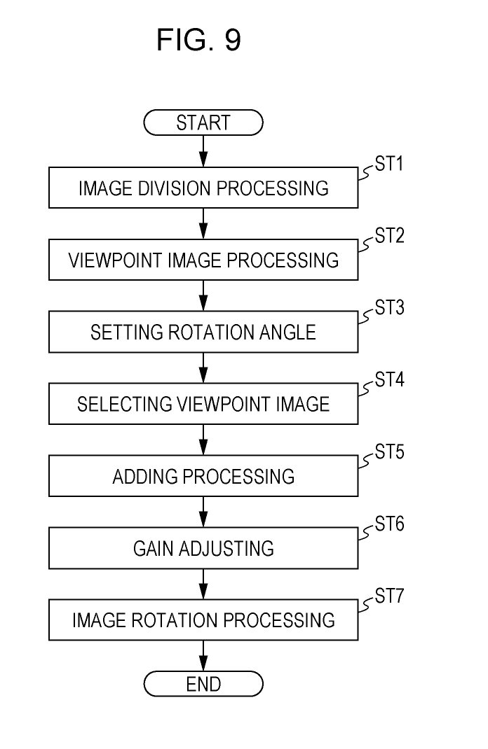

Subsequently, an image processing operation in an endoscope device will be described. FIG. 9 is a flowchart which illustrates a part of image processing operations in the endoscope device.

When light beam information is generated, an endoscope device 10 performs image division processing in step ST1. The endoscope device 10 generates an image signal of a viewpoint image in each viewpoint by dividing the light beam information in each viewpoint in each microlens, and proceeds to step ST2.

In step ST2, the endoscope device 10 performs viewpoint image processing. The endoscope device 10 performs signal processing of an image signal in each viewpoint image, and proceeds to step ST3.

In step ST3, the endoscope device 10 sets a rotation angle. The endoscope device 10 sets a rotation angle by selecting any one of a rotation angle which is set according to a user operation, a rotation angle with respect to an initial position, a rotation angle with respect to the gravity direction, and a rotation angle which is detected by image matching, and proceeds to step ST4.

In step ST4, the endoscope device 10 selects a viewpoint image. The endoscope device 10 reads out image selection information corresponding to a set rotation angle from the table, or calculates image selection information in each setting of rotation angle, and selects a viewpoint image which is used when generating an image signal of a left eye image, and a viewpoint image which is used when generating an image signal of a right eye image based on the image selection information.

In step ST5, the endoscope device 10 performs adding processing. The endoscope device 10 adds the viewpoint image which is selected for generating the left eye image, and generates an image signal of the left eye image. In addition, the endoscope device 10 adds the viewpoint image which is selected for generating the right eye image, generates an image signal of the right eye image, and proceeds to step ST6.

In step ST6, the endoscope device 10 performs gain adjusting. The endoscope device 10 performs gain adjusting of an image signal of the left eye image, or the right eye image according to the number of viewpoint images to be added when generating the left eye image and right eye image. That is, the endoscope device 10 removes an influence due to a difference in the number of added viewpoint images by increasing gain according to the number of added viewpoint images are decreased, and proceeds to step ST7.

In step ST7, the endoscope device 10 performs image rotation processing. The endoscope device 10 rotates the generated left eye image and right eye image to a direction corresponding to the rotation angle.

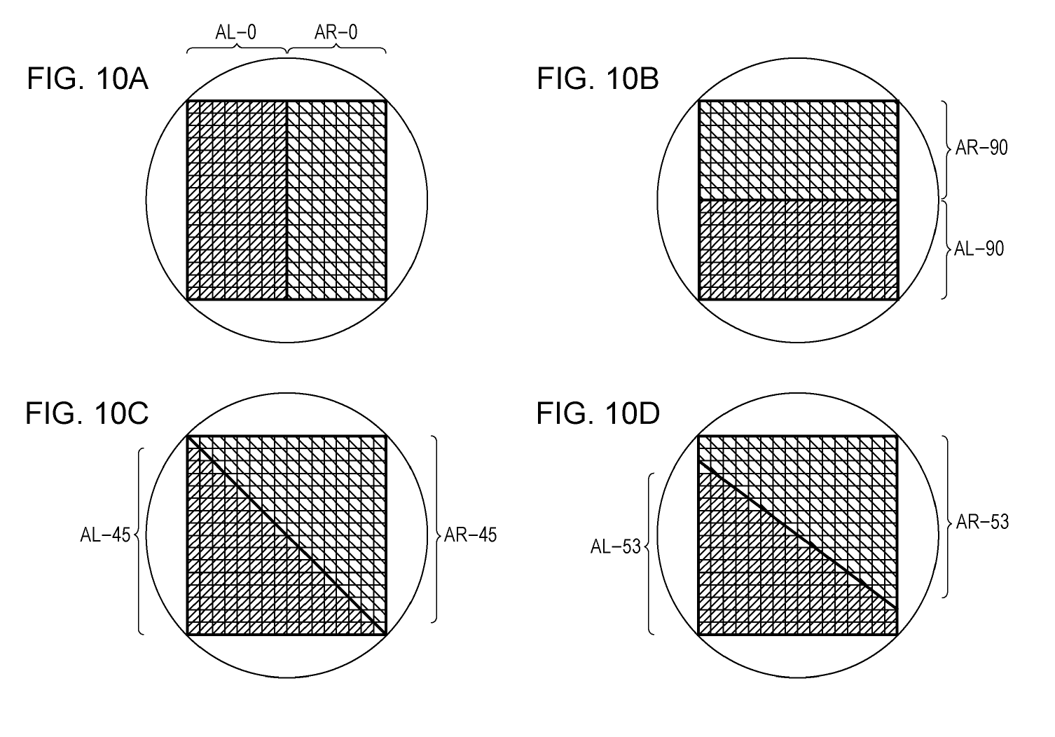

Subsequently, the image processing operation in the endoscope device will be described in detail. FIGS. 10A to 10D illustrate relationships between rotation angle and a viewpoint image which is selected in the image selection unit. In addition, the image selection table 611 of the image selection unit 61 stores image selection information which denotes a viewpoint image which is selected according to a rotation angle. In addition, in FIGS. 10A to 10D, cases in which the number of viewpoints is “256” are illustrated.

When the rotation angle is “0°”, as illustrated in FIG. 10A, the image selection unit 61 selects a viewpoint image of a viewpoint which is included in the region AL-0, and outputs the viewpoint image to the addition processing unit 71L, selects a viewpoint image of a viewpoint which is included in the region AR-0, and outputs the viewpoint image to the addition processing unit 71R.

When the rotation angle is “90°”, as illustrated in FIG. 10B, the image selection unit 61 selects a viewpoint image of a viewpoint which is included in the region AL-90, and outputs the viewpoint image to the addition processing unit 71L, selects a viewpoint image of a viewpoint which is included in the region AR-90, and outputs the viewpoint image to the addition processing unit 71R.

When the rotation angle is “45°”, as illustrated in FIG. 10C, the image selection unit 61 selects a viewpoint image of a viewpoint which is included in the region AL-45, and outputs the viewpoint image to the addition processing unit 71L, selects a viewpoint image of a viewpoint which is included in the region AR-45, and outputs the viewpoint image to the addition processing unit 71R.

When the rotation angle is “53°”, as illustrated in FIG. 10D, the image selection unit 61 selects a viewpoint image of a viewpoint which is included in the region AL-53, and outputs the viewpoint image to the addition processing unit 71L, selects a viewpoint image of a viewpoint which is included in the region AR-53, and outputs the viewpoint image to the addition processing unit 71R.

In addition, in FIGS. 10C and 10D, viewpoints with no hatching denote viewpoint images which are not used when generating the left eye image and right eye image.

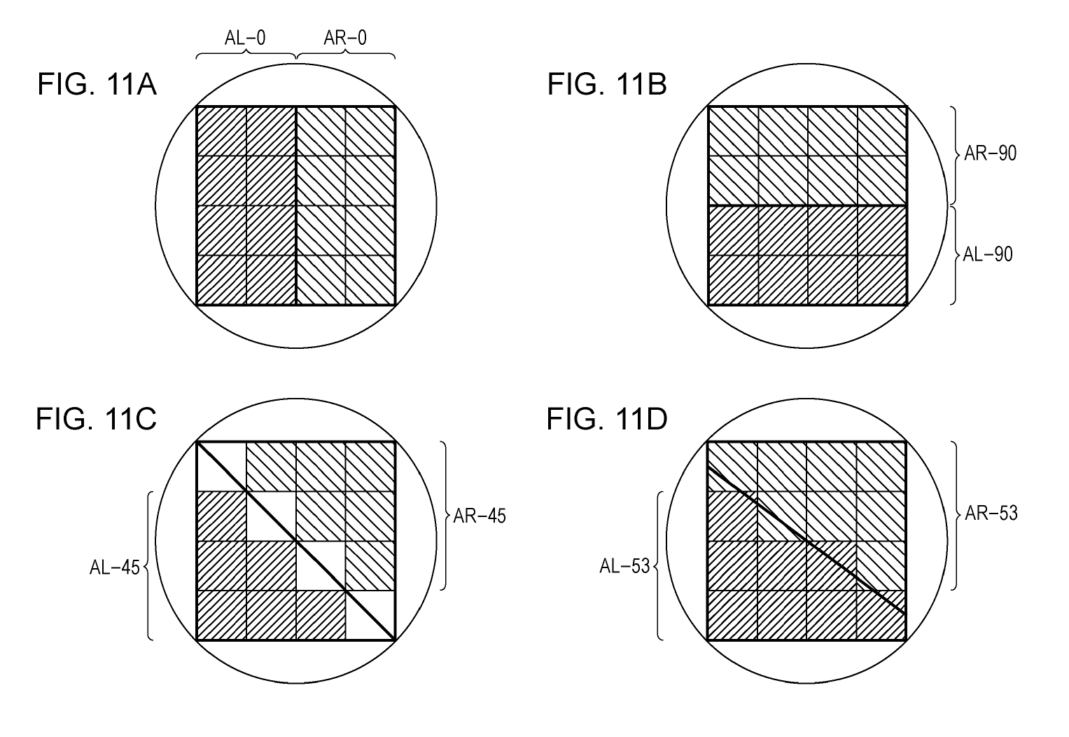

FIGS. 11A to 11d illustrate cases in which the number of viewpoints is “16”. When the rotation angle is “0°”, as illustrated in FIG. 11A, the image selection unit 61 selects a viewpoint image of a viewpoint which is included in the region AL-0, and outputs the viewpoint image to the addition processing unit 71L, selects a viewpoint image of a viewpoint which is included in the region AR-0, and outputs the viewpoint image to the addition processing unit 71R.

When the rotation angle is “90°”, as illustrated in FIG. 11B, the image selection unit 61 selects a viewpoint image of a viewpoint which is included in the region AL-90, and outputs the viewpoint image to the addition processing unit 71L, selects a viewpoint image of a viewpoint which is included in the region AR-90, and outputs the viewpoint image to the addition processing unit 71R.

When the rotation angle is “45°”, as illustrated in FIG. 11C, the image selection unit 61 selects a viewpoint image of a viewpoint which is included in the region AL-45, and outputs the viewpoint image to the addition processing unit 71L, selects a viewpoint image of a viewpoint which is included in the region AR-45, and outputs the viewpoint image to the addition processing unit 71R.

When the rotation angle is “53°”, as illustrated in FIG. 11D, the image selection unit 61 selects a viewpoint image of a viewpoint which is included in the region AL-53, and outputs the viewpoint image to the addition processing unit 71L, selects a viewpoint image of a viewpoint which is included in the region AR-53, and outputs the viewpoint image to the addition processing unit 71R.

In this manner, when selecting a viewpoint image according to a rotation angle, a left eye image and right eye image which are generated by being added with a selected viewpoint image are images in which viewpoints are rotated around the optical axis of the imaging optical system 22.

In addition, when a viewpoint image is selected according to a rotation angle, and is added, if the number of viewpoint images to be added is small, a signal level of the image after adding becomes small. Therefore, the gain adjusting units 72L and 72R perform the gain adjusting according to the number of viewpoint images to be added. Therefore, in cases illustrated in FIGS. 10A and 10B, the number of viewpoints included in the regions AL-0, AR-0, AL-90, and AR-90 is “128”. In this case, since the number of whole viewpoints is “256”, the gain adjusting unit 72L makes the image signal of the left eye image (256/128) times, and the gain adjusting unit 72R makes the image signal of the right eye image, for example, (256/128) times.

In addition, in a case of FIG. 10C, the number of viewpoints included in the regions AL-45, and AR-45 is “120”. Accordingly, the gain adjusting unit 72L makes the image signal of the left eye image (256/120) times, and the gain adjusting unit 72R makes the image signal of the right eye image, for example, (256/120) times.

Further, in a case illustrated in FIG. 10D, the number of viewpoints included in the regions AL-53, and AR-53 is “125”. Accordingly, the gain adjusting unit 72L makes the image signal of the left eye image (256/125) times, and the gain adjusting unit 72R makes the image signal of the right eye image, for example, (256/125) times.

By performing such gain adjusting, the image signals of the left eye image and right eye image become image signals in which the influence due to a difference in the number of viewpoint images to be added is removed.

Meanwhile, the left eye image and right eye image which are generated in the addition processing units 71L and 71R are images in which the viewpoints are rotated around the optical axis of the imaging optical system 22 according to the rotation angle, however, subject images in the left eye image and right eye image are not in a rotated state. Accordingly, the rotation processing units 74L and 74R rotate the direction of the left eye image and right eye image according to a rotation angle so that the subject images become images which are rotated according to the rotation angle.

For example, as illustrated in FIG. 10B, when the rotation angle is “90°”, the left eye image and right eye image becomes images in which the viewpoint and the subject images are rotated according to the rotation angle by rotating the left eye image and right eye image by “90°”, respectively, around the optical axis.

Accordingly, according to the first embodiment, it is possible to generate the left eye image and right eye image corresponding to a rotation angle without mechanically rotating the pupil division prism, or the two imaging elements. For this reason, it is possible to miniaturize an endoscope. In addition, since it is not necessary to mechanically rotate the imaging elements or the like, there is little malfunction, and an adjustment with high precision is not necessary. Further, calibration for compensating an influence due to an assembling error in a portion of a device, a secular change, a change in temperature, or the like is also not necessary.

In addition, a configuration of generating a viewpoint image or generating a left eye image and right eye image, and performing adjusting may be provided, for example, at a grip portion or the like in the rigid endoscope, or the flexible endoscope, and may be provided at a processing unit 91 in a capsule endoscope.

### 2. Second Embodiment

Meanwhile, in the first embodiment, a case in which the image processing device according to the present technology is installed in an endoscope has been described. However, the image processing device according to the present technology may be separately provided from the endoscope. Subsequently, in a second embodiment, a case in which the image processing device is separately provided from an endoscope will be described.

### 2-1. Configuration of Endoscope

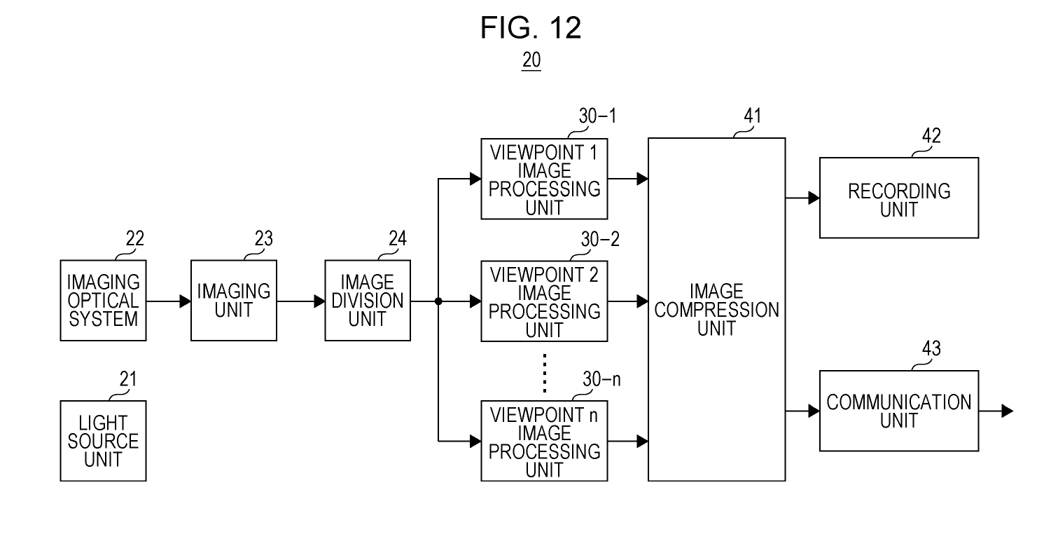

FIG. 12 illustrates a configuration example of an endoscope in which the image processing device according to the present technology is not provided. An endoscope 20 includes a light source unit 21, an image processing system 22, an imaging unit 23, an image division unit 24, a viewpoint 1 image processing unit 30-1 to viewpoint n image processing unit 30-n, an image compression unit 41, a recording unit 42, and a communication unit 43.

The light source unit 21 emits illumination light to an observation target. The imaging optical system 22 is configured by a focus lens, a zoom lens or the like, and causes an optical image of the observation target to which the illumination light is radiated (subject optical image) to be formed as an image in the imaging unit 23.

In the imaging unit 23, a light field camera which is able to record light beam information (light field data) which also includes channel information (direction of input light) of input light, not only light quantity information of the input light is used. The light field camera is provided with a microlens array 230 immediately front of an image sensor 231 such as a CCD, or a CMOS as described above, generates light beam information including light quantity information and channel information of input light, and output the light beam information to the image division unit 24.

The image division unit 24 divides the light beam information which is generated in the imaging unit 23 in each viewpoint, and generates image signals of a plurality of viewpoint images. For example, the image signal of the viewpoint 1 image is generated, and is output to the viewpoint 1 image processing unit 30-1. Similarly, the image signal of the viewpoint 2 (to n) image is generated, and is output to the viewpoint 2 (to n) image processing unit 30-2 (to n).

The viewpoint 1 image processing unit 30-1 to viewpoint n image processing unit 30-n perform the same image processing as that in the first embodiment with respect to image signals of viewpoint images which are supplied from the image division unit 24, and outputs the image signal of the viewpoint image after the image processing to the image compression unit 41.

The image compression unit 41 compresses a signal amount by performing encoding processing of the image signal of each viewpoint image. The image compression unit 41 supplies an encoded signal which is obtained by performing the encoding processing to the recording unit 42, or the communication unit 43. The recording unit 42 records the encoded signal which is supplied from the image compression unit 41 in a recording medium. The recording medium may be a recording medium which is provided in the endoscope 20, or may be a detachable recording medium. The communication unit 43 generates a communication signal using the encoded signal which is supplied from the image compression unit 41, and transmits the signal to an external device through a wired, or wireless transmission path. The external device may be the image processing device of the present technology, or may be a server device, or the like.

### 2-2. Operation of Endoscope

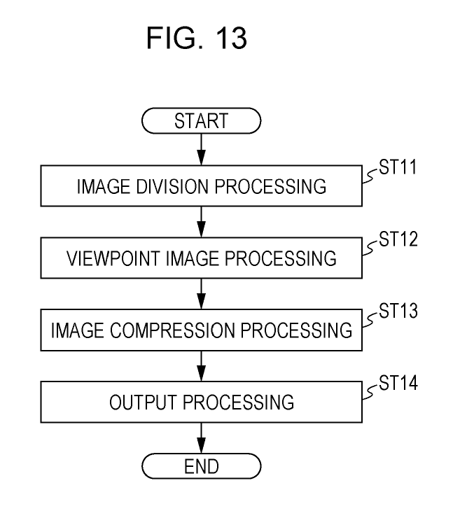

Subsequently, an operation in the endoscope will be described. FIG. 13 is a flowchart which illustrates a part of the operation of the endoscope.

When light beam information is generated in the endoscope 20, in step ST11, the endoscope 20 performs image dividing processing. The endoscope 20 generates an image signal of a viewpoint image in each viewpoint by dividing light beam information in each viewpoint in each microlens, and proceeds to ST12.

In step ST12, the endoscope 20 performs viewpoint image processing. The endoscope 20 performs signal processing of an image signal in each viewpoint image, and proceeds to step ST13.

In step ST13, the endoscope 20 performs image compression processing. The endoscope 20 performs encoding processing with respect to image signals of a plurality of viewpoint images, generates an encoded signal in which a signal amount is compressed, and proceeds to step ST14.

In step ST14, the endoscope 20 performs output processing. The endoscope 20 performs processing of outputting the encoded signal which is generated in step ST13, for example, recording the generated encoded signal in a recording medium, or transmitting the encoded signal to an external device as a communication signal.

The endoscope 20 performs the above described processing, and records an image signal of a viewpoint image which is input to the image selection unit 61 in the first embodiment in the recording medium, or transmits to the external device in a state in which the image signal is encoded.

### 2-3. Configuration of Image Processing Device

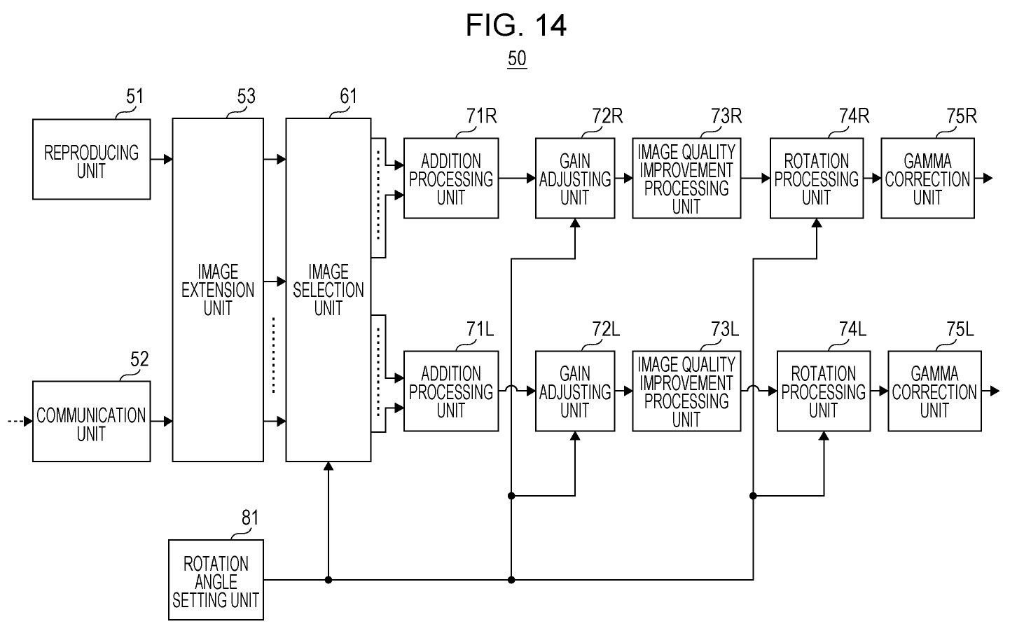

FIG. 14 illustrates a configuration example of an image processing device. An image processing device 50 includes a reproducing unit 51, a communication unit 52, and an image extension unit 53. In addition, the image processing device 50 further includes an image selection unit 61, addition processing units 71L and 71R, gain adjusting units 72L and 72R, image quality improvement processing units 73L and 73R, rotation processing units 74L and 74R, gamma correction units 75L and 75R, and viewpoint rotation angle setting unit 81.

The reproducing unit 51 reads out an encoded signal of a viewpoint image from a recording medium, and outputs the signal to the image extension unit 53.

The communication unit 52 receives a communication signal which is transmitted through a wired, or wireless transmission path from the endoscope 20, or an external device such as a server. In addition, the communication unit 52 outputs the encoded signal which is transmitted through the communication signal to the image extension unit 53.

The image extension unit 53 performs decoding processing of the encoded signal which is supplied from the reproducing unit 51, or the communication unit 52. The image extension unit 53 outputs image signals of the plurality of viewpoint images which are obtained by performing the decoding processing to the image selection unit 61.

The image selection unit 61 selects a viewpoint image according to a viewpoint rotation angle from the plurality of viewpoint images having different viewpoints. The image selection unit 61 sets a plurality of viewpoint regions, for example, viewpoint regions of a left eye image and viewpoint regions of a right eye image based on the rotation angle which is set in the viewpoint rotation angle setting unit 81, and selects a viewpoint image of a viewpoint which is included in the set viewpoint region in each region. The image selection unit 61 outputs a viewpoint image of a viewpoint which is included in a viewpoint region of a left eye image to the addition processing unit 71L, and outputs a viewpoint image of a viewpoint which is included in a viewpoint region of a right eye image to the addition processing unit 71R.

The addition processing unit 71L generates an image signal of a left eye image by adding a viewpoint image which is supplied from the image selection unit 61. The addition processing unit 71L outputs the image signal of the left eye image which is obtained by performing the addition processing to the gain adjusting unit 72L. The addition processing unit 71R generates an image signal of a right eye image by adding a viewpoint image which is supplied from the image selection unit 61. The addition processing unit 71R outputs the image signal of the right eye image which is obtained by performing the addition processing to the gain adjusting unit 72R.

The gain adjusting unit 72L performs gain adjusting corresponding to a rotation angle with respect to an image signal of the left eye image. As described above, the image signal of the left eye image is generated by adding the image signal of the viewpoint image which is selected in the image selection unit 61 in the addition processing unit 71L. Accordingly, when the number of viewpoint images which are selected in the image selection unit 61 is small, a signal level of the image signal becomes small. For this reason, the gain adjusting unit 72L adjusts gain according to the number of viewpoint images which are selected in the image selection unit 61, and removes an influence due to a difference in the number of viewpoint images to be added. The gain adjusting unit 72L outputs the image signal after the gain adjusting to image quality improvement processing unit 73L.

The gain adjusting unit 72R performs gain adjusting corresponding to a rotation with respect to an image signal of the right eye image. The gain adjusting unit 72R adjusts gain according to the number of viewpoint images which are selected in the image selection unit 61, similarly to the gain adjusting unit 72L, and removes an influence due to a difference in the number of viewpoint images to be added. The gain adjusting unit 72R outputs the image signal after the gain adjusting to image quality improvement processing unit 73R.

The image quality improvement processing unit 73L performs high resolution of an image using classification adaptation processing or the like. For example, the image quality improvement processing unit 73L generates an image signal with high resolution by improving sharpness, contrast, color, or the like. The image quality improvement processing unit 73L outputs the image signal after the image quality improvement processing to a rotation processing unit 74L. The image quality improvement processing unit 73R performs the high resolution of an image using the classification adaptation processing or the like, similarly to the image quality improvement processing unit 73L. The image quality improvement processing unit 73R outputs the image signal after the image quality improvement processing to a rotation processing unit 74R.

The rotation processing unit 74L rotates the left eye image. The rotation processing unit 74L performs rotation processing based on a rotation angle with respect to the left eye image which is generated in the addition processing unit 71L, and then is subjected to the gain processing, or the image quality improvement processing, and rotates the direction of the left eye image. The rotation processing unit 74L outputs the image signal of the rotated left eye image to the gamma correction unit 75L. The rotation processing unit 74R rotates the right eye image. The rotation processing unit 74R performs rotation processing based on a rotation angle with respect to the right eye image, and rotates the direction of the right eye image. The rotation processing unit 74R outputs the image signal of the rotated right eye image to the gamma correction unit 75R.

The gamma correction unit 75L performs correction processing based on gamma characteristics of a display device which performs an image display of an imaged image with respect to the left eye image, and outputs the image signal of the left eye image which is subjected to the gamma correction to an external display device, or the like. The gamma correction unit 75R performs correction processing based on gamma characteristics of a display device which performs an image display of an imaged image with respect to the right eye image, and outputs the image signal of the right eye image which is subjected to the gamma correction to the external display device, or the like.

The viewpoint rotation angle setting unit 81 informs the image selection unit 61, and the rotation processing units 74L and 74R of a rotation angle by setting the rotation angle according to a user operation or the like.

2-4. Operation of image processing device

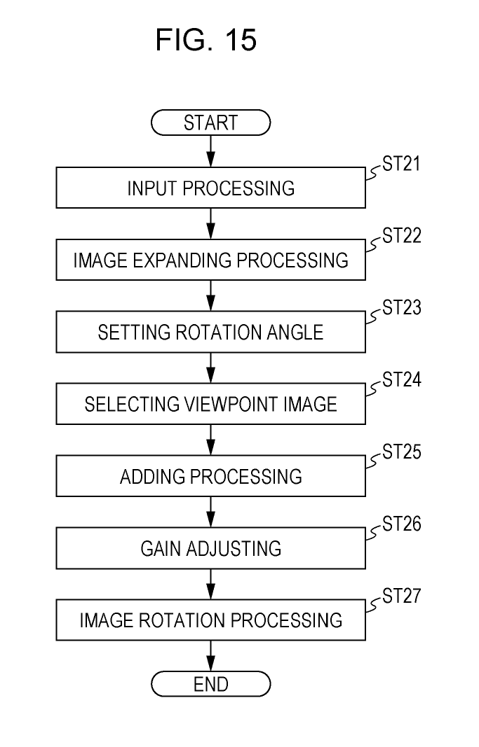

FIG. 15 is a flowchart which illustrates an operation of the image processing device. In step ST21, the image processing device 50 performs input processing. The image processing device 50 reads out an encoded signal which is generated in the endoscope 20 from a recording medium. In addition, the image processing device 50 obtains the encoded signal which is generated in the endoscope 20 from the endoscope 20, or an external device such as a server through a wired, or a wireless transmission path, and proceeds to step ST22.

In step ST22, the image processing device 50 performs image extending processing. The image processing device 50 performs decoding processing of the encoded signal which is read out from the recording medium, or the encoded signal which is received from the endoscope 20, or the like, generates image signals of a plurality of viewpoint images, and proceeds to step ST23.

In step ST23, the image processing device 50 sets a rotation angle. The image processing device 50 sets the rotation angle according to, for example, a user operation, or the like, and proceeds to step ST24.

In step ST24, the image processing device 50 selects a viewpoint image. The image processing device 50 reads out image selection information corresponding to a rotation angle from a table, and selects a viewpoint image which is used for generating an image signal of a left eye image, and a viewpoint image which is used for generating an image signal of a right eye image based on the read out image selection information.

In step ST25, the image processing device 50 performs adding processing. The image processing device 50 adds the viewpoint image which is selected for generating the left eye image, and generates an image signal of the left eye image. In addition, the image processing device 50 adds the viewpoint image which is selected for generating the right eye image, and generates an image signal of the right eye image, and proceeds to step ST26.

In step ST26, the image processing device 50 performs gain adjusting. When generating the left eye image and right eye image, the image processing device 50 performs gain adjusting of the image signal of the left eye image, or the right eye image according to the number of viewpoint images to be added. That is, the image processing device 50 sets gain high when the number of viewpoint images to be added becomes small, removes an influence due to a difference in the number of viewpoint images to be added, and proceeds to step ST27.

In step ST27, the image processing device 50 performs image rotation processing. The image processing device 50 rotates the generated left eye image and right eye image to the direction corresponding to a rotation angle.

In such a second embodiment, the endoscope and the image processing device are separately configured, and image signals of the plurality of viewpoint images are supplied to the image processing device from the endoscope through a recording medium, or a transmission path. Accordingly, an observer is able to obtain the same left eye image and right eye image as those in a case of performing imaging using an instructed rotation angle only by instructing a rotation angle with respect to the image processing device. In addition, the observer is able to easily perform observing of a subject even if imaging of the subject is not performed by controlling a rotation angle using the endoscope. Further, since the observer is able to rotate a viewpoint by performing an operation with respect to the image processing device, an operator of the endoscope is not necessary to consider an imaging angle when imaging a subject, and may perform an operation so that a desired subject can be imaged well. Accordingly, it is possible to reduce a burden of the operator of the endoscope.

### 3. Other Embodiments

Meanwhile, in the above described first and second embodiments, a case in which a viewpoint is rotated around the optical axis has been described, however, it is possible to generate further various images when a region of viewpoint images which is selected in order to generate an image with a new viewpoint is controlled. In addition, in other embodiments, the endoscope 10 of which configuration is denoted in the first embodiment may be used, or the image processing device 50 of which configuration is denoted in the second embodiment may be used.

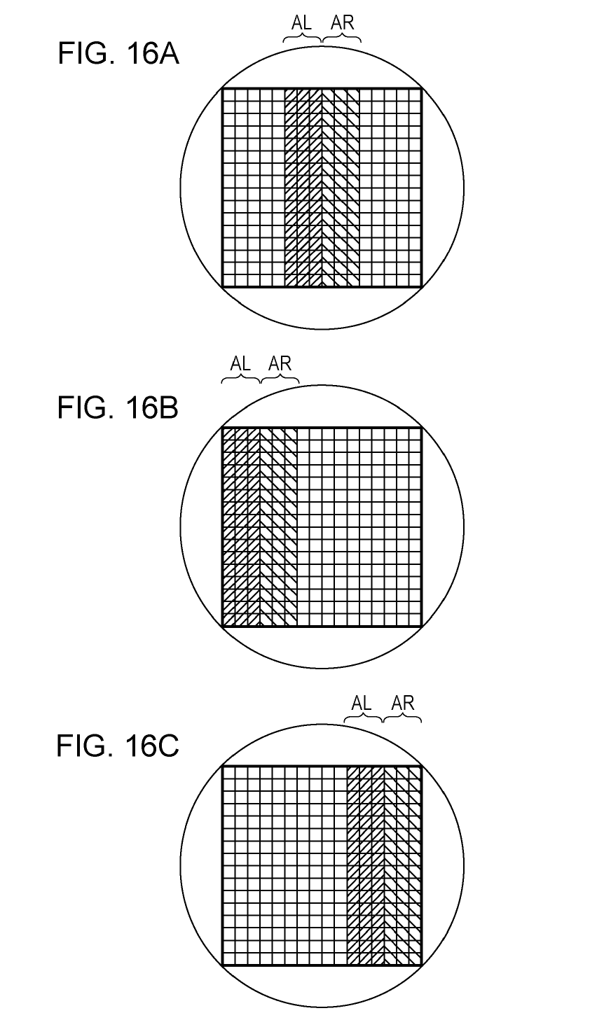

Subsequently, as other embodiments, a case in which a viewpoint is moved in the horizontal direction (corresponding to case in which viewpoint position is rotated in horizontal direction when seen from imaged subject, for example, imaged subject at center) will be described. FIGS. 16A to 16C illustrate operations when viewpoints are moved in the horizontal direction. For example, as illustrated in FIG. 16A, the image selection unit 61 sets a region AL of a predetermined range to the left from the center, selects viewpoint images of viewpoints which are included in the region AL, and outputs image signals of the selected viewpoint images to the addition processing unit 71L. In addition, the image selection unit 61 sets a region AR of a predetermined range to the right from the center, selects viewpoint images of viewpoints which are included in the region AR, and outputs image signals of the selected viewpoint images to the addition processing unit 71R.

When the viewpoint is moved to the left direction, as illustrated in FIG. 16B, the image selection unit 61 shifts the predetermined regions AL and AR to the left direction based on the rotation angle (horizontal direction). In addition, the image selection unit 61 selects viewpoint images in viewpoints which are included in the region AL, and outputs image signals of the selected viewpoint images to the addition processing unit 71L. In addition, the image selection unit 61 selects viewpoint images in viewpoints which are included in the region AR, and outputs image signals of the selected viewpoint images to the addition processing unit 71R.

When the viewpoint is moved to the right direction, as illustrated in FIG. 16C, the image selection unit 61 shifts the predetermined regions AL and AR to the right direction based on the rotation angle. In addition, the image selection unit 61 selects viewpoint images in viewpoints which are included in the region AL, and outputs image signals of the selected viewpoint images to the addition processing unit 71L. In addition, the image selection unit 61 selects viewpoint images in viewpoints which are included in the region AR, and outputs image signals of the selected viewpoint images to the addition processing unit 71R.

In this manner, the image selection unit 61 is able to move a viewpoint in a stereoscopic vision in the horizontal direction by selecting viewpoint images by shifting the regions AL and AR according to the rotation angle (horizontal direction). In addition, as illustrated in FIGS. 10A to 10D, when an operation of selecting a viewpoint image is performed by combination based on a rotation angle (rotation angle around optical axis), it is possible to move a viewpoint also in the vertical direction, or in the oblique direction, not just in the horizontal direction.

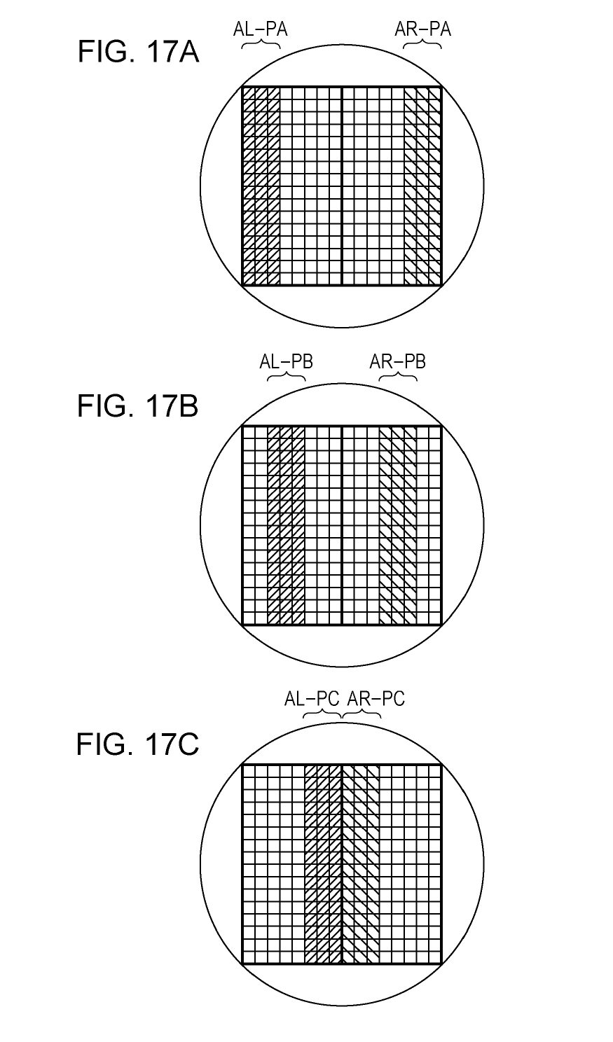

Further, a selection of a viewpoint image may also be performed based on other information, not just based on a rotation angle. FIGS. 17A to 17C exemplify operations when parallax adjusting is performed. The image selection table 611 of the image selection unit 61 outputs image selection information corresponding to parallax adjusting information to the matrix switching unit 612. For example, when an instruction of setting a maximum parallax is made in the parallax adjusting information, as illustrated in FIG. 17A, the image selection unit 61 selects viewpoint images of viewpoints which are included in the region AL-PA which is a predetermined range from the left end, and outputs image signals of the selected viewpoint images to the addition processing unit 71L. In addition, the image selection unit 61 selects viewpoint images of viewpoints which are included in the region AR-PA which is a predetermined range from the right end, and outputs image signals of the selected viewpoint images to the addition processing unit 71R.

When setting a parallax smaller than a maximum parallax based on the parallax adjusting information, as illustrated in FIG. 17B, the image selection unit 61 selects viewpoint images of viewpoints which are included in the region AL-PB which is a predetermined range shifted to the center from the left end, and outputs image signals of the selected viewpoint images to the addition processing unit 71L. In addition, the image selection unit 61 selects viewpoint images of viewpoints which are included in the region AR-PB which is a predetermined range shifted to the center from the right end, and outputs image signals of the selected viewpoint images to the addition processing unit 71R.

When setting a minimum parallax based on parallax adjusting information, as illustrated in FIG. 17C, the image selection unit 61 selects viewpoint images of viewpoints which are included in the region AL-PC which is a predetermined range from the center, and outputs image signals of the selected viewpoint images to the addition processing unit 71L. In addition, the image selection unit 61 selects viewpoint images of viewpoints which are included in the region AR-PC which is a predetermined range from the center, and outputs image signals of the selected viewpoint images to the addition processing unit 71R.

In this manner, when the gap between two regions is adjusted based on the parallax adjusting information, a parallax between a left eye image and right eye image becomes large since the left eye image and right eye image are generated by adding viewpoint images of viewpoints which are separated from the center when the parallax is set to be large. In addition, when the parallax is set to be small, the parallax between the left eye image and right eye image becomes small since the left eye image and right eye image are generated by adding viewpoint images of viewpoints which are close to the center. In this manner, by adjusting the gap between two regions, it is possible to make the parallax between the left eye image and right eye image as a desired parallax amount.

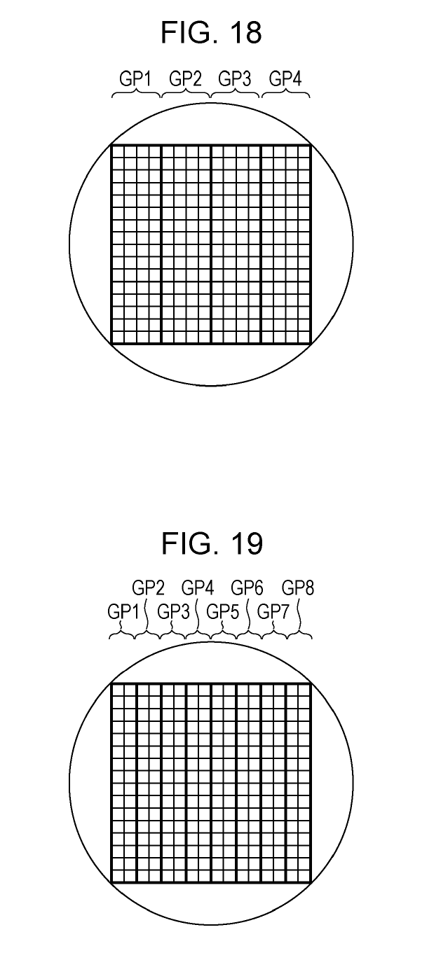

FIG. 18 illustrates a case in which viewpoints are divided into four groups, and FIG. 19 illustrates a case in which viewpoints are divided into eight groups. As illustrated in FIG. 18, when boundaries of the groups are provided in the vertical direction, and are divided into four groups of GP1 to GP4, an image to which a viewpoint image of a viewpoint included in the group GP1 is added becomes an image of which a viewpoint is located on the left side of an image to which a viewpoint image of a viewpoint which is included in the group GP2 which is close to the right side of the group GP1 is added. Similarly, an image to which a viewpoint image of a viewpoint included in the group GP4 is added becomes an image of which a viewpoint is located on the right side of an image to which a viewpoint image of a viewpoint which is included in the group GP3 which is close to the left side of the group GP4 is added. In addition, an image to which a viewpoint image of a viewpoint included in the group GP2 is added becomes an image of which a viewpoint is moved to the left side of the center, since the group GP2 is located on the left side of the center. Further, an image to which a viewpoint image of a viewpoint included in the group GP3 is added becomes an image of which a viewpoint is moved to the right side of the center, since the group GP3 is located on the right side of the center. Accordingly, as illustrated in FIG. 18, when the viewpoints are divided into four groups, it is possible to generate four images of which viewpoint positions are different in the horizontal direction.

In addition, as illustrated in FIG. 19, when viewpoints are made into eight groups of GP1 to GP8, it is possible to generate eight images of which viewpoint positions are different in the horizontal direction. Accordingly, when performing switching of groups to which viewpoint images are added, it is possible to easily generate a left eye image and right eye image of which viewpoints are different.

In addition, in FIGS. 18 and 19, cases in which boundaries of groups are provided in the vertical direction have been exemplified, however, when the boundaries of the groups are provided in the horizontal direction, it is possible to generate images of which viewpoint positions are different in the vertical direction. In addition, the boundaries of the groups may be provided in the oblique direction. In this manner, when viewpoints are made into a plurality of groups, it is possible for them to be used in a display of naked eye stereoscopic vision, or the like.

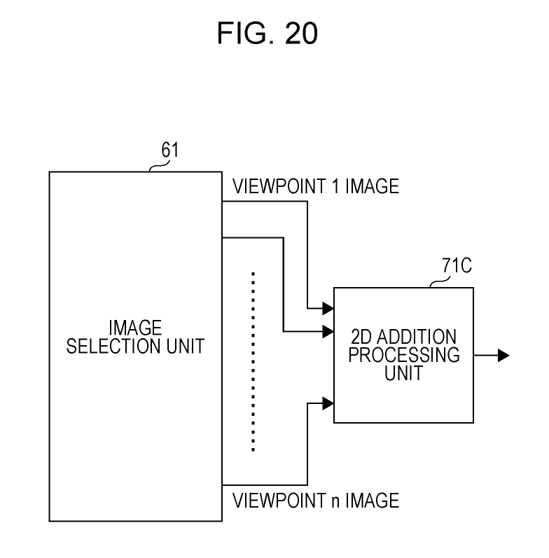

Further, the endoscope 10, or the image processing device 50 may generate an image to which all of the viewpoint images are added. That is, by adding all of the viewpoint images, the same 2D image as the image which is generated based on a light beam input to each microlens, or the image which is generated using an imaging device in the related art in which an imaging element is provided at a position of the microlens is generated. Accordingly, when a 2D addition processing unit 71C which is illustrated in FIG. 20 is provided in the endoscope 10, or the image processing device 50, it is possible to generate an image signal of a 2D image, not only the image signals of the left eye image and right eye image.

In addition, the generation of image signal of a 2D image is not limited to a case in which all of the viewpoints are added. For example, it is possible to generate an image signal of a 2D image even when viewpoint images of all of viewpoints which are in the same distance from a separation point are added. Specifically, it is possible to generate a 2D image even when viewpoint images of viewpoints which are included in the region AL-PA, and the region AR-PA illustrated in FIG. 17A are added as all of the viewpoint images of viewpoints which are in the same distance from the separation point. In addition, it is also possible to generate a 2D image even when viewpoint images of viewpoints which are included in the regions AL-PC, and AR-PC illustrated in FIG. 17C are added. In this case, since viewpoint images with small parallaxes are added compared to a case in which the viewpoint images of viewpoints which are included in the regions AL-PA, and AR-PA are added, the 2D image is rarely influenced by the parallax. Further, when moving the regions AL-PC and AR-PC in combination according to a rotation angle, it is possible to generate a 2D image in which viewpoints are moved according to the rotation angle.

In addition, the above described series of image processing may be executed using software, hardware, or a combination of both the software and hardware. When the processing is executed using software, a program in which a processing sequence is recorded is installed to a memory in a computer which is incorporated in dedicated hardware, and is executed. Alternately, it is possible to execute by installing a program to a general-purpose computer in which various processing can be executed.

For example, the program can be recorded in advance in a hard disk, or a ROM (Read Only Memory) as a recording medium. Alternately, the program can be temporarily, or permanently stored (recorded) in a removable recording medium such as a flexible disk, a CD-ROM (Compact Disc Read Only Memory), a MO (Magneto Optical) disc, a DVD (Digital Versatile Disc), a magnetic disk, a semiconductor memory card. Such a removable recording medium can be provided as so-called package software.

In addition, the program may be installed to a computer from a removable recording medium, and may be transmitted to a computer in a wireless, or a wired manner through a network, such as a LAN (Local Area Network), or the Internet from a download site. The computer may receive the program which is transmitted in such a manner, and install the program in a recording medium such as an embedded hard disk.

In addition, the present technology is not interpreted by being limited to the above described embodiments. The embodiments of the present technology disclose the technology by exemplifying thereof, and as a matter of course, those skilled in the art can perform modifications, or substitutions of the embodiments without departing from the scope of the present technology. That is, in order to determine the scope of the present technology, it is necessary to consider the claims.

In addition, the image processing device according to the present technology may have the following configuration.

(1) An image processing device which includes an image selection unit which selects a viewpoint image according to a viewpoint rotation angle from a plurality of viewpoint images having different viewpoints; and an addition processing unit which generates a viewpoint image with a new viewpoint by adding a viewpoint image which is selected in the image selection unit.

(2) The image processing device which is disclosed in (1), in which the image selection unit sets a plurality of viewpoint regions according to the viewpoint rotation angle, and selects a viewpoint image of a viewpoint which is included in the set viewpoint regions, and the addition processing unit adds a viewpoint image to each of the viewpoint regions.

(3) The image processing device which is disclosed in (2), in which the image selection unit sets a viewpoint region of a left eye image, and a viewpoint region of a right eye image according to the viewpoint rotation angle, and the addition processing unit generates a left eye image and a right eye image by adding a viewpoint image to each of the viewpoint regions.

(4) The image processing device which is disclosed in (3), in which the image selection unit controls a gap between the viewpoint region of the left eye image and the viewpoint region of the right eye image, and adjusts a parallax amount of the left eye image and right eye image.

(5) The image processing device which is disclosed in (3), or (4), in which the image selection unit selects all of viewpoint images, or viewpoint images of viewpoints which are included in viewpoint regions of the left eye image and right eye image, and the addition processing unit generates a plan image by adding the viewpoint images which are selected in the image selection unit.

(6) The image processing device which is disclosed in any one of (1) to (5), further includes a gain adjusting unit which performs gain adjusting corresponding to the number of viewpoint images which is added with respect to the viewpoint image with the new viewpoint.

(7) The image processing device which is disclosed in (6), in which the gain adjusting unit sets gain high when the number of added viewpoint images becomes small.

(8) The image processing device which is disclosed in any one of (1) to (7), further includes a rotation processing unit which performs image rotation processing according to the viewpoint rotation angle with respect to the viewpoint image with the new viewpoint.

(9) The image processing device which is disclosed in any one of (1) to (8), further includes an imaging unit which generates light beam information including channel information and light quantity information of a light beam which is input through an imaging optical system, and an image division unit which generates the plurality of viewpoint images having different viewpoints from the light beam information which is generated in the imaging unit.

(10) The image processing device which is disclosed in (9), further includes a viewpoint rotation angle setting unit which sets the viewpoint rotation angle, in which the viewpoint rotation angle setting unit sets an angle of an imaging unit with respect to any one of a gravity direction, or an initial direction, an angle in which an image which is imaged in the imaging unit becomes an image which is the most similar to a reference image when being rotated, or an angle which is designated by a user is set to the viewpoint rotation angle.

(11) The image processing device which is disclosed in any one of (1) to (10), further includes an image decoding unit which performs decoding processing of an encoding signal which is generated by performing encoding processing of a plurality of viewpoint images of which the viewpoints are different, in which the image decoding unit outputs image signals of the plurality of viewpoint images including different viewpoints which are obtained by performing decoding processing of an encoded signal to the image selection unit.

The present disclosure contains subject matter related to that disclosed in Japanese Priority Patent Application JP 2012-059736 filed in the Japan Patent Office on Mar. 16, 2012, the entire contents of which are hereby incorporated by reference.

What is claimed is:

1. An image processing device comprising:
an image selection unit which selects a viewpoint image according to a viewpoint rotation angle from a plurality of viewpoint images having different viewpoints; and
an addition processing unit which generates a viewpoint image with a new viewpoint by adding a viewpoint image which is selected in the image selection unit.

2. The image processing device according to claim 1,
wherein the image selection unit sets a plurality of viewpoint regions according to the viewpoint rotation angle, and selects a viewpoint image of a viewpoint which is included in the set viewpoint regions, and
wherein the addition processing unit adds a viewpoint image to each of the viewpoint regions.

3. The image processing device according to claim 2,
wherein the image selection unit sets a viewpoint region of a left eye image, and a viewpoint region of a right eye image according to the viewpoint rotation angle, and
wherein the addition processing unit generates a left eye image and a right eye image by adding a viewpoint image to each of the viewpoint regions.

4. The image processing device according to claim 3,
wherein the image selection unit controls a gap between the viewpoint region of the left eye image and the viewpoint region of the right eye image, and adjusts a parallax amount of the left eye image and the right eye image.

5. The image processing device according to claim 3,
wherein the image selection unit selects all of viewpoint images, or viewpoint images of viewpoints which are included in viewpoint regions of the left eye image and the right eye image, and
wherein the addition processing unit generates a plan image by adding the viewpoint images which are selected in the image selection unit.

6. The image processing device according to claim 1, further comprising:
a gain adjusting unit which performs gain adjusting corresponding to the number of viewpoint images which is added with respect to the viewpoint image with the new viewpoint.

7. The image processing device according to claim 6,
wherein the gain adjusting unit sets gain high when the number of added viewpoint images becomes small.

8. The image processing device according to claim 1, further comprising:
a rotation processing unit which performs image rotation processing according to the viewpoint rotation angle with respect to the viewpoint image with the new viewpoint.

9. The image processing device according to claim 1, further comprising:
an imaging unit which generates light beam information including channel information and light quantity information of a light beam which is input through an imaging optical system; and
an image division unit which generates the plurality of viewpoint images having different viewpoints from the light beam information which is generated in the imaging unit.

10. The image processing device according to claim 9, further comprising:
a viewpoint rotation angle setting unit which sets the viewpoint rotation angle,
wherein the viewpoint rotation angle setting unit sets an angle of an imaging unit with respect to any one of a gravity direction, or an initial direction, an angle in which an image which is imaged in the imaging unit becomes an image which is the most similar to a reference image when being rotated, or an angle which is designated by a user is set to the viewpoint rotation angle.

11. The image processing device according to claim 1, further comprising:
an image decoding unit which performs decoding processing of an encoding signal which is generated by performing encoding processing of a plurality of viewpoint images of which the viewpoints are different,
wherein the image decoding unit outputs image signals of the plurality of viewpoint images including different viewpoints which are obtained by performing decoding processing of an encoded signal to the image selection unit.

12. An image processing method comprising:
selecting a viewpoint image according to a viewpoint rotation angle from a plurality of viewpoint images having different viewpoints; and
generating a viewpoint image with a new viewpoint by adding the selected viewpoint image.
